(19) United States
(12) Patent Application Publication
(10) Pub. No.: US 2022/0386863 A1
(43) Pub. Date: Dec. 8, 2022

(54) SMART CONTACT LENS FOR DIAGNOSIS AND TREATMENT OF DRY EYE SYNDROME

(71) Applicants: PHI BIOMED INC., Seoul (KR); POSTECH RESEARCH AND BUSINESS DEVELOPMENT FOUNDATION, Gyeongsangbuk-do (KR)

(72) Inventors: Sei Kwang Hahn, Seoul (KR); Sang Baie Shin, Gyeongsangbuk-do (KR); Jong Hwan Mun, Seoul (KR); Tae Yeon Kim, Gyeongsangbuk-do (KR)

(21) Appl. No.: 17/765,431

(22) PCT Filed: Feb. 24, 2021
(86) PCT No.: PCT/KR2021/002304

§ 371 Date: Mar. 30, 2022

(30) Foreign Application Priority Data
Feb. 24, 2020 (KR) 10-202-00022250

(51) Int. Cl. A61B 3/10; A61B 5/00; A61F 9/00; A61B 5/1477

(52) U.S. Cl. CPC A61B 3/101; A61B 5/6821; A61B 5/4839; A61F 9/0017; A61B 5/1477

(57) ABSTRACT

The present invention relates to a contact lens for diagnosis and treatment of dry eye syndrome. The contact lens, according to the present invention, may diagnose dry eye syndrome by means of a change in an electric current occurring as a result of a field effect when a dry eye syndrome biomarker binds to a probe, and when dry eye syndrome is diagnosed, generates an electric current so that a gold film blocking a drug reservoir melts by the electric current, thereby enabling the release of a drug to be controlled.

### TECHNICAL FIELD

The present invention relates to a smart contact lens for diagnosing and treating a dry eye syndrome.

### BACKGROUND ART

A dry eye syndrome refers to an ocular disease in which an ocular surface is damaged due to lack of tears, excessive evaporation of tears, or imbalance in tear composition. Generally, the dry eye syndrome causes irritation symptoms such as eye irritation soreness, irritation, foreign body sensation, dryness, and the like. Such a dry eye syndrome causes not only simple tear deficiency but also eye discomfort due to inflammation in tears and an ocular surface (cornea and conjunctiva), amblyopia, and instability of a tear layer to cause damage to the ocular surface. In addition, the dry eye syndrome is highly likely to induce pain, irregular corneal surface, blurred and fluctuating vision, and diseases such as corneal ulcer. Although the pathogenesis of the dry eye syndrome has not been fully elucidated, it is reported that inflammation, which is related to infiltration of inflammatory cells, increased expression of immune-activating molecules and adhesion molecules, Th1 and Th17 responses, abnormal changes in apoptotic markers and chemokines, and the like play, plays an important role.

In general, the intake of foods rich in potassium and anthocyanins is recommended for the treatment of a dry eye syndrome. In addition, the intake of fruits such as kale, kiwi, apple, or the like is highly recommended. Furthermore, a drop of artificial tears or a treatment, which blocks lacrimal points to regulate a secretion amount of tears, is often used. However, a dry eye syndrome is a common disease occurring in around 20% of Korean adults. In particular, the number of dry eye syndrome patients is continuously increasing due to a temperature rise caused by a global climate change, especially, the El Nino phenomenon, and environmental pollution, but there is no special treatment or functional food.

That is, a dry eye syndrome is an eye disease that greatly affects a visual function and the quality of life. However, it takes a long time for symptoms to appear, and an accurate diagnosis is difficult due to the lack of diagnostic methods. Therefore, there is a need for new methods of diagnosing a dry eye syndrome, and among the methods, there is a need for technologies for diagnosing a dry eye syndrome using a dry eye syndrome biomarker present in tears.

### DISCLOSURE

### Technical Problem

The present invention is directed to providing a smart contact lens for diagnosing and treating a dry eye syndrome.

### Technical Solution

According to an embodiment of the present invention, a contact lens for diagnosing and treating a dry eye syndrome includes a dry eye syndrome biomarker detecting sensor, and a drug reservoir.

The dry eye syndrome biomarker detecting sensor and the drug reservoir may be formed on a transparent substrate.

### Advantageous Effects

A contact lens for diagnosing and treating a dry eye syndrome according to the present invention can diagnose a dry eye syndrome through a change in current caused by an electric field effect when a dry eye syndrome biomarker is coupled to a probe. In addition, when the dry eye syndrome is diagnosed, an electrical signal can be generated, and a gold film blocking a drug reservoir can be dissolved by a current according to the electrical signal to adjust the release of a drug. Therefore, the contact lens according to the present invention can be effectively used for diagnosing and treating a dry eye syndrome.

### DESCRIPTION OF DRAWINGS

FIG. 1 is a schematic diagram of a field effect transistor biosensor according to the present invention.

FIG. 2 shows results of measuring a change in current caused when a biomarker and an aptamer are coupled according to the present invention.

FIG. 3 shows images showing a structure of a drug reservoir according to the present invention.

FIG. 4 shows a patterned transparent and flexible substrate according to the present invention.

FIG. 5 shows a change in current due to an electrochemical reaction of a gold thin film according to the present invention.

FIG. 6 shows a model drug released from a drug reservoir according to the present invention.

### BEST MODES OF THE INVENTION

The present invention relates to a contact lens for diagnosing and treating a dry eye syndrome, which includes a dry eye syndrome biomarker detecting sensor and a drug reservoir.

[symbol]

Hereinafter, the contact lens for diagnosing and treating a dry eye syndrome of the present invention will be described in more detail.

A dry eye syndrome refers to an ocular disease in which an ocular surface is damaged due to lack of tears, excessive evaporation of tears, or imbalance in tear composition. Generally, the dry eye syndrome causes irritation symptoms such as eye irritation soreness, irritation, foreign body sensation, dryness, and the like. In the present invention, by using the contact lens for diagnosing and treating a dry eye syndrome, a dry eye syndrome may be diagnosed through a change in current caused by an electric field effect when a dry eye syndrome biomarker is coupled to a probe. In addition, when the dry eye syndrome is diagnosed, an electrical signal may be generated, and a gold film blocking the drug reservoir may be dissolved according to the electrical signal to release a dry eye syndrome treatment drug.

The contact lens of the present invention may is based on at least one polymer selected from the group consisting of an elastomer such as a silicone elastomer, a silicone hydrogel, and a polymer hydrogel of poly(2-hydroxyethyl methacrylate) (PHEMA), polyvinylpyrrolidone (PVP), poly(lactic acid-glycolic acid) (PLGA), or polyvinyl alcohol (PVA).

In the present invention, a transparent substrate is formed inside the contact lens, and the dry eye syndrome biomarker detecting sensor and the drug reservoir may be formed on the transparent substrate.

In one embodiment, the dry eye syndrome biomarker detecting sensor and the drug reservoir may be formed on a surface of the transparent substrate which faces an eyeball.

The transparent substrate has characteristics such as excellent light transmittance, excellent flexibility and elasticity, and excellent biocompatibility. The transparent substrate may include at least one selected from the group consisting of parylene C, polydimethylsiloxane (PDMS), a silicone elastomer, polyethylene terephthalate (PET), and polyimide (PI).

In the present invention, the dry eye syndrome biomarker detecting sensor may serve to detect a dry eye syndrome biomarker present in tears. Since the dry eye syndrome biomarker detecting sensor is directly worn on an eye in a state of being included in the contact lens, the dry eye syndrome biomarker detecting sensor is suitable for detecting a dry eye syndrome biomarker present in tears.

In one embodiment, the dry eye syndrome biomarker may be at least one selected from the group consisting of a vascular endothelial growth factor (VEGF), lactoferrin, and lipocalin.

In the present invention, the dry eye syndrome biomarker detecting sensor may be a field effect transistor (FET) biosensor.

An “FET”” is a transistor in which a current of a source and a drain is controlled using a principle of applying a voltage to a gate electrode to form a channel (gate) through which electrons or holes flow by an electric field of the channel.

In addition, a “FET biosensor” includes a source electrode and a drain electrode at both sides of a substrate and a gate which is in contact with the source and drain electrodes and is formed on the substrate. The FET biosensor is a sensor in which, since a probe is fixed to a surface of the gate or a surface of the electrode, when a target material is coupled to the probe, a change in current caused during the coupling is measured through an electrical method to detect whether the probe and the target material are coupled.

In the present invention, the FET biosensor is applied to the contact lens to detect a change in current caused when the dry eye syndrome biomarker coupled to the probe, thereby diagnosing a dry eye syndrome.

The FET biosensor according to the present invention may include a source electrode and a drain electrode which are formed on the transparent substrate and are spaced apart from each other by a certain distance,

a FET channel which connects the source electrode and the drain positive electrode, and

a probe attached onto the channel.

In one embodiment, the source electrode and the drain electrode may each independently include a gold thin film or nanomaterials, and the nanomaterials may be materials having biocompatibility. Specifically, the nanomaterials may include at least one selected from the group consisting of zero-dimensional materials that are nanoparticles, one-dimensional nanomaterials that are nanowires, nanofibers, or nanotubes, and two-dimensional nanomaterials that are graphene, MoS₂, or nanoflakes. More specifically, the electrode may include silver (Ag) and/or gold (Au) and may include a gold thin film or silver nanowires.

In one embodiment, the FET channel may connect the source electrode and the drain electrode and may be a semi-conductive layer. Specifically, the FET channel may include at least one selected from the group consisting of one-dimensional materials including silicon nanowires and two-dimensional materials including graphene or MoS₂. More specifically, the FET channel may include graphene or MoS₂.

In addition, in one embodiment, the probe may be coupled to the channel and may be coupled to the dry eye syndrome biomarker. The probe may be an aptamer or an antibody coupled to the dry eye syndrome biomarker. For example, when the dry eye syndrome biomarker is a VEGF, a VEGF aptamer may be used as the probe. When the dry eye syndrome biomarker is lactoferrin, at least one selected from the group consisting of Ylac1, Ylac4, Ylac5, Ylac6, Ylac8, and Ylac9 may be used for the probe. When the dry eye syndrome biomarker is lipocalin, at least one selected from the group consisting of NA10, NA36, NA42, NA53, and NA21 may be used for the probe.

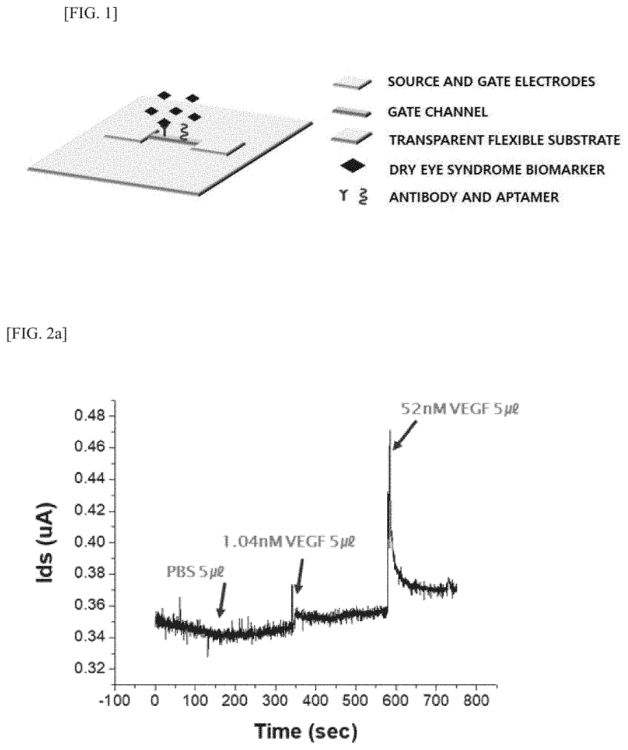

In the present invention, FIG. 1 is a schematic diagram of the FET biosensor according to an example of the present invention.

As shown in FIG. 1, the source electrode and the drain electrode are formed on a transparent flexible substrate, and the two electrodes may be connected through the FET transistor channel (gate channel). The probe that is an antibody or an aptamer may be coupled onto the channel, and the dry eye syndrome biomarker may be coupled to the probe.

In the present invention, the drug reservoir is sealed by an electrode pattern including gold and may be linked with the above-described FET biosensor. When a dry eye syndrome is diagnosed by the FET biosensor, an electrical signal may be generated, and the gold of the electrode pattern of the drug reservoir may be dissolved by reacting with chlorine ions in a living body according to the electrical signal, thereby releasing a drug from the drug reservoir.

In one embodiment, in the dry eye syndrome biomarker detecting sensor, when the biomarker is coupled to the probe, results of measuring a change in current change caused by an electric field effect is transmitted to an external system (specifically, smart glasses) through wireless communication. When the received results of measuring the change in current is analyzed to detect an abnormality in the external system (that is, when a dry eye syndrome is diagnosed), an electrical signal is transmitted to the drug reservoir. The drug reservoir may be driven through the received electrical signal.

In one embodiment, the drug reservoir may include the electrode pattern including gold and formed on a portion of a surface of the transparent substrate and a drug well layer formed on the electrode pattern and including one or more drug wells with a shape that is recessed to face outward. In this case, perforations may be formed in the transparent substrate, and the electrode pattern may surround the perforations.

In one embodiment, a drug may be positioned in the drug well. The drug may be a drug capable of treating a dry eye syndrome or may include a drug carrier which may release the drug capable of treating the dry eye syndrome, and a drug-release-controlling material. In addition, the drug may have a structure of micelles or nanoparticles or may exist in a state of being coupled to a polymer.

In the present invention, an antenna may be additionally formed on the transparent substrate in addition to the above-described FET biosensor and drug reservoir. The antenna may be formed to be coplanar with the FET biosensor on the transparent substrate.

The antenna may transmit and receive power and signals to and from the outside through induced current and electromagnetic resonance.

In one embodiment, the antenna may be a circular antenna having a circular structure.

In one embodiment, the antenna may be made of nanomaterials, and the nanomaterials may include at least one selected from the group consisting of metal thin film materials, zero-dimensional materials that are nanoparticles, one-dimensional nanomaterials that are nanowires, nanofibers, or nanotubes, and two-dimensional nanomaterials that are graphene, MoS₂, or nanoflakes.

Components constituting the FET biosensor, the drug reservoir, and the antenna according to the present invention all have excellent biocompatibility and thus may operate stably in tears and may be harmless to a living body.

[symbol]

In addition, the present invention relates to a method of manufacturing the above-described contact lens for diagnosing and treating a dry eye syndrome.

The contact lens for diagnosing and treating a dry eye syndrome may be manufactured through step S1 of forming a sacrificial layer soluble in water on a handling substrate,

step S2 of forming a transparent substrate on the sacrificial layer,

step S3 of forming a FET biosensor and a drug reservoir on the transparent substrate, and

step S4 of transferring the transparent substrate, on which a dry eye syndrome biomarker detecting sensor and the drug reservoir are formed, into the contact lens.

Step S1 is a step of forming the sacrificial layer on the handling substrate.

The sacrificial layer may serve as an adhesive layer between the handling substrate and the transparent substrate and may assist in transferring the transparent substrate on which the dry eye syndrome biomarker detecting sensor and the drug reservoir are formed. As long as the sacrificial layer is soluble in water, the sacrificial layer is not particularly limited and may include at least one selected from the group consisting of PVA, dextran, and the like

Step S2 is a step of forming the transparent substrate on the sacrificial layer. Since the sacrificial layer serves as an adhesive, the transparent substrate may be easily attached to the handling substrate and may be easily delaminated from the handling substrate by dissolving the sacrificial layer in a subsequent process.

In one embodiment, a material having excellent light transmittance may be used for the transparent substrate, and the above-described type may be used therefor.

Step S3 is a step of forming the dry eye syndrome biomarker detecting sensor and the drug reservoir on the transparent substrate. The dry eye syndrome biomarker detecting sensor may be the FET biosensor.

In one embodiment, the FET biosensor may be manufactured through step a1 of forming a mask material for patterning on the transparent substrate,

step a2 of patterning, through photolithography as a source electrode and a drain electrode, nanomaterials on the transparent substrate on which the mask material is formed,

step a3 of forming a FET channel between the patterned source and drain electrodes, and

step a4 of coupling a probe onto the formed FET channel.

Step a1 is a step of forming the mask material for patterning on the transparent substrate.

The mask material may serve as a shadow mask, and the mask material may be used to pattern the nanomaterials. As the mask material, a material usable as a photoresist may be used without limitation.

Step a2 is a step of patterning, through photolithography as the source electrode and the drain electrode, the nanomaterials on the transparent substrate, on which the mask material is formed.

Through the above step, a pattern of the nanomaterials may be formed on the transparent substrate. The above-described types may be used as the nanomaterials, and specifically, a gold thin film or silver nanowires may be used.

The nanomaterials patterned in the above step may be used for the source electrode and the drain electrode.

Step a3 is a step of forming the FET channel between the patterned source electrode and drain electrode.

In the above step, an oxidized surface is patterned on the transparent substrate through a photolithography method in the form of a channel connecting the electrodes, and silver nanowires are deposited on the oxidized surface to form the channel.

In addition, step a4 is a step of coupling the probe onto the formed FET channel.

In the above step, an amide group introduced onto the channel may be coupled to the probe.

In one embodiment, the drug reservoir may be manufactured through step b1 of forming an electrode pattern including gold on a portion of a surface of the transparent substrate,

step b2 of forming a drug well layer including one or more drug wells on the electrode pattern, and

step b3 of forming a drug release channel in the transparent substrate.

Step b1 is a step of forming the electrode pattern including gold on the portion of the surface of the transparent substrate.

In the present invention, the electrode pattern may be formed in a form in which a gold electrode pattern is formed on the transparent substrate using photolithography and positive electrodes of the electrode pattern surround the drug reservoir.

In one embodiment, the electrode pattern stacked on the transparent substrate may include the positive electrodes made of a metal including gold and a negative electrode commonly connected to the positive electrodes. The electrodes may have a form in which a plurality of positive electrodes form an array. The electrode may be made of gold and titanium according to portions thereof. The gold forming the electrode pattern may be removed by being electrolyzed by a voltage applied in an electrolyte. Therefore, the positive electrode of the electrode pattern may be used as a gate of a channel (drug release channel) through which an accommodated drug is delivered by a voltage.

Step b2 is a step of forming the drug well layer including one or more drug weds on the electrode pattern. The drug well may store a drug.

The drug well layer including the drug wells may include a flexible and biocompatible component. Specifically, the drug well layer may include at least one selected from the group consisting of PDMS, a silicone elastomer, polyurethane acrylate (PUA), and an SU8. The drug well layer may be formed on the electrode pattern.

Step b3 is a step of forming the drug release channel in the transparent substrate.

In step b3, the drug release channel may allow a gold electrode to react with chlorine ions in a living body, and when the gold electrode is dissolved through an electrochemical reaction, the drug release channel may serve as a channel though which a drug is released. The drug release channel may be formed by forming a perforation in the transparent substrate, and in this case, the perforation may be formed through reactive ion etching (RIE) using oxygen.

In addition, step S4 is a step of transferring the transparent substrate, on which the dry eye syndrome biomarker detecting sensor and the drug reservoir are formed, into the contact lens.

The sensor and the drug reservoir formed on the sacrificial layer may be transferred by dissolving the sacrificial layer in biocompatible water.

In addition, the method of the present invention may further include a step of forming an antenna on the transparent substrate. The above step may be performed during step S3.

[symbol]

In addition, the present invention relates to a system for diagnosing and treating a dry eye syndrome.

The system for diagnosing and treating a dry eye syndrome of the present invention may include a contact lens, which includes a dry eye syndrome biomarker detecting sensor and a drug reservoir, and smart glasses.

As the contact lens, the above-described contact lens may be used. Specifically, in the contact lens, the dry eye syndrome biomarker detecting sensor and the drug reservoir are formed on a transparent substrate. The dry eye syndrome biomarker detecting sensor may diagnose a dry eye syndrome through a change in current caused by an electric field effect when a biomarker is coupled to a probe. When the dry eye syndrome is diagnosed, an electrical signal may be generated, and a gold film of the drug reservoir may be dissolved according to the electrical signal to release a drug of the drug reservoir.

In the present invention, the smart glasses may wirelessly transmit or receive an electrical signal to control the driving of the drug reservoir of the contact lens which is wirelessly driven.

In one embodiment, the dry eye syndrome biomarker detecting sensor and the drug reservoir may be connected to an application-specific integrated circuit (ASIC) chip to enable wireless communication. A result detected by the sensor may be transmitted to an external system, that is, the smart glasses, to store and process data, and the driving of a drug delivery system may be controlled. The drug reservoir may be driven by receiving an electrical signal transmitted from the external system through the ASIC chip.

Specifically, when the drug reservoir receives an electrical signal transmitted from the smart glasses, and gold of an electrode pattern is dissolved in chlorine ions in a living body to become AuCl⁴⁻ according to the electrical signal, the electrode pattern may open so that a drug may be released to the outside from the drug reservoir.

The present invention may provide the smart glasses which enable micro-unit long distance adjustment using a transparent electrode including nanomaterials, stretchable electronics, a complementary metal-oxide semiconductor (CMOS), a flexible and biocompatible micro electro-mechanical system (MEMS), and nano electro-mechanical system (NEMS) technology.

In the smart glasses, electrical power may be implemented using wireless inductive power transfer (WiTricity) technology, and wireless communication may be performed using Bluetooth, infrared (IR), and radio frequency (RF) communications in the smart glasses.

An operating system (OS) of the smart glasses may use an Android OS, and the smart glasses may be equipped with an OMAP 4430 SoC, a dual-core central processing unit (CPU), and a 4 GB random-access memory (RAM). A display screen may include 640×360 pixels, and a bone conduction transducer may be used for a sound. An optical sensor, a biosensor, a pressure, a temperature, and functions of an acoustic electromagnetic (EM) sensor may be controlled using a voice through a microphone, and the smart glasses may be paired with a smartphone, a smart watch, or a personal computer (PC). An embedded 100 mAh lithium ion battery may be used for power, and a photocell may be inserted for auto powering. A total weight of the smart glasses may be less than 20 g, and Wi-Fi 802.11 b/g, Bluetooth, and micro Universal Serial Bus (USB) may be available. Photos with a resolution of more than 15 MP and videos with a format of more than 720 p may be implemented using a mounted camera.

[symbol]

In addition, the present invention relates to a method of diagnosing and treating a dry eye syndrome using the above-described system.

In the method of diagnosing and treating a dry eye syndrome according to the present invention, a dry eye syndrome biomarker detecting sensor in a contact lens detects a biomarker in tears of a subject at a predetermined measurement time. When the biomarker is detected, an electrical signal may be generated, and gold of an electrode pattern sealing a drug well of a drug reservoir may be dissolved in chlorine ions according to the electrical signal to become AuCl⁴⁻. Thus, the drug reservoir may open.

In one embodiment, the dry eye syndrome biomarker detecting sensor may detect the biomarker and transmit a result of a change in current, which is caused when the biomarker and a probe are coupled, to smart glasses through radio frequency (RF) wireless communication.

In addition, in one embodiment, a drug delivery system is driven through an electrical signal transmitted from the smart glasses. When the smart glasses analyze the change in current received through the sensor and detect an abnormality, the smart glasses may transmit an electrical signal to the drug reservoir. When a gold electrode pattern sealing the drug reservoir of the drug delivery system receives the signal, the gold electrode pattern may be dissolved in chlorine ions to become AuCl⁴⁻, and thus, the drug delivery reservoir may open.

In addition, in one embodiment, power generated from a wireless electric coil of the smart glasses may be received by a wireless electric antenna of the contact lens which is wirelessly driven, and the received power may be used to drive the sensor and the drug delivery system under control of an integrated circuit (IC) chip.

### Modes of the Invention

Hereinafter, the present invention will be described in detail through the following Examples. However, the following Examples are for illustrative purposes only, and the present invention is not intended to be limited by the following Examples.

[symbol]

### EXAMPLES

### Example 1: Manufacture of FET Sensor using Graphene

A FET sensor was manufactured using a principle in which a dry eye syndrome is diagnosed through a change in current flowing in a circuit due to a change in charge distribution which is caused when a biomarker is coupled to a probe.

Electrodes using a gold thin film and silver nanowires were patterned on a silicon substrate using a photolithography method, and a semi-conductive layer was placed between the electrodes. In this case, silver nanowires were used as a material of the semi-conductive layer. Thereafter, an aptamer (anti-VEGF aptamer) capable of being coupled to a dry eye syndrome biomarker was coupled to the semi-conductive layer.

Specifically, a silver nanowire channel was immersed in an 3-aminopropyldimethyl-ethoxysilane (APDES)/ethanol solution to introduce an amine group, and the silver nanowire channel was immersed in a sodium carbonate buffer including 1 wt % succinic anhydride to substitute the amine group with a carboxyl group. Then, after the silver nanowire channel is immersed in a phosphate buffered saline (PBS) solution, 1-ethyl-3(3-dimethyl aminopropyl)carbodiimide hydro-chloride (EDC) and sulfa N-hydroxysuccinimide (sulfa-NHS) were added to form an amide group. An aptamer solution was introduced into the channel, into which the amide group was introduced, to couple the channel and the probe.

[symbol]

### Experimental Example 1. Observation of Change in Current Generated when Biomarker and Aptamer are coupled

(1) Method

A VEGF was injected into the sensor in real time to monitor an intensity of a current flowing in the silver nanowire channel.

Specifically, 200 μL of PBS was dropped onto the FET sensor to set a base current for 30 minutes, and then, a change in current generated by injecting a specific concentration of a VEGF was observed.

[symbol]

(2) Results

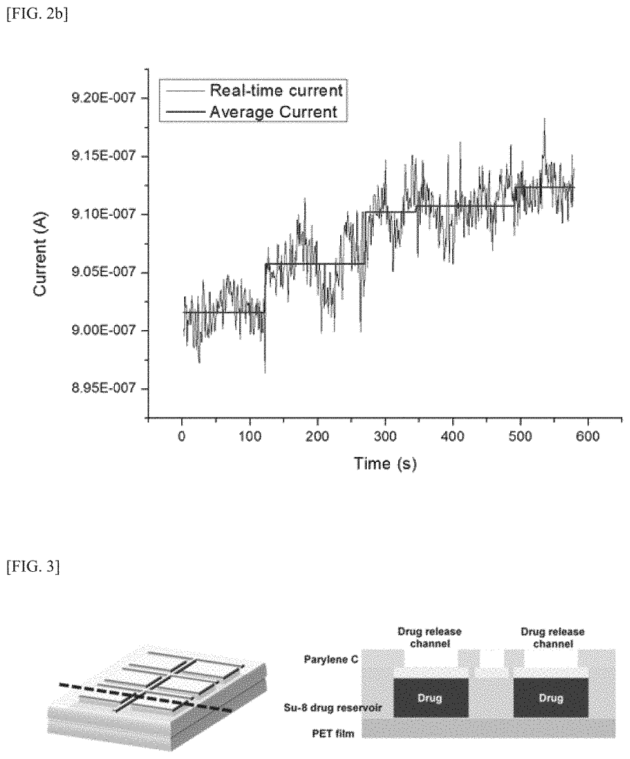

Results of the observation are shown in FIG. 2.

As shown in FIG. 2A, it can be confirmed that there is no difference in current when 104 pM of s VEGF is injected, but a large change is observed when 1.04 nM and 52 nM of a VEGF are injected.

In FIG. 2B, it is observed that, as a VEGF is injected, a current suddenly increases and then gradually stabilizes. When currents are averaged for each section, it can be confirmed that a stabilized current after the VEGF is injected is also higher than an initial base current.

[symbol]

### Example 2. Manufacture of Drug Delivery System

A handling substrate was spin-coated with PVS to form a sacrificial layer, and a parylene layer was applied on the sacrificial layer. Gold eras deposited to a thickness of 100 nm on a manufactured transparent substrate, and patterning was performed using photolithography to form an electrode.

A drug well layer including one or more drug wells was formed on the electrode through photolithography using an SU8, and a drug was mixed into the drug wells and loaded.

Thereafter, a drug reservoir was sealed using a PET film and a parylene layer was deposited to perform passivation (see FIG. 3).

Then, the sacrificial layer was dissolved in water, and then a manufactured drug delivery system was separated from the handling substrate and transferred.

In order to form a channel through which the drug is releasable, on the transparent substrate, a photoresist was formed in a shape of a drug release channel through RIE or laser cutting. After perforations were formed through RIE using oxygen, the photoresist was removed using acetone.

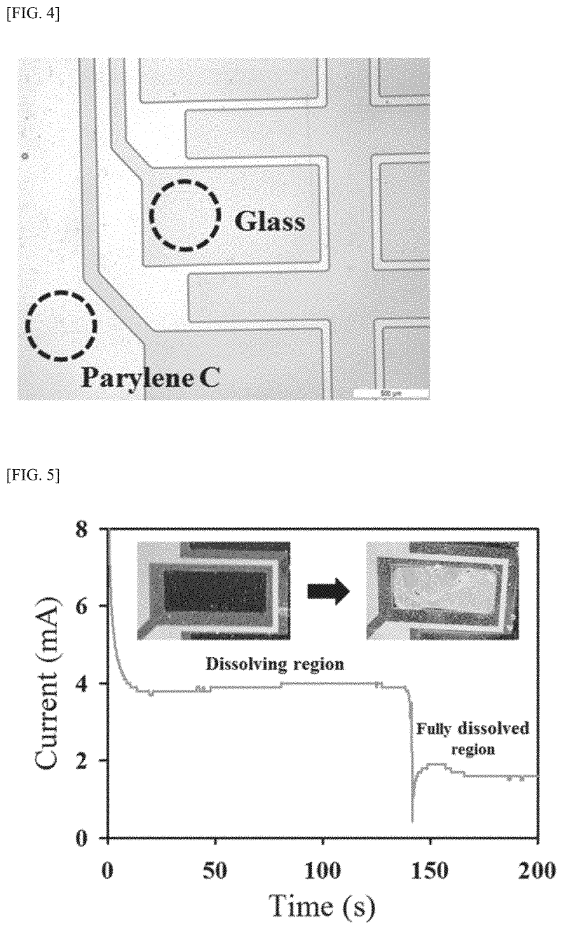

In an etched portion of the patterned substrate, a gold thin film was exposed to the outside to react with chlorine ions present in a body to cause an electrochemical reaction (see FIG. 4).

[symbol]

In the present invention, FIG. 3 shows images showing a structure of a drug reservoir. An electrode structure includes positive electrodes and a negative electrode, and it can be confirmed that a drug well (drug reservoir and drug release channel) is covered with a gold thin film.

In addition, FIG. 4 is an image of a drug release channel formed by patterning a transparent substrate through RIE.

[symbol]

### Example 3. Manufacture of Contact Lens for Diagnosing and Treating Dry Eye Syndrome

A transparent substrate, on which a FET sensor and a drug reservoir were formed, was manufactured. The sensor and the drug reservoir were formed on the transparent substrate according to methods of Examples 1 and 2.

The transparent substrate was introduced into a lens manufacturing mold which contained a contact lens manufacturing solution, and constant pressure was applied to perform heat treatment in an oven at about 100° C. for one hour. A contact lens manufactured in the lens manufacturing mold was removed.

The manufactured contact lens includes the sensor and the drug reservoir.

[symbol]

### Experimental Example 2. Release of Drug according to Diagnosis of Dry Eye Syndrome

(1) Method

When the drug reservoir was manufactured, 5 mg/ml of rhodamine B that is a model drug was loaded into the drug reservoir and dried to then manufacture a drug delivery system.

After PBS is dropped, a voltage of 1.8 V was applied between a positive electrode and a negative electrode using a potentiostat, and a change in current was measured as a gold thin film was dissolved.

[symbol]

(2) Results

FIG. 5 shows a change in current due to an electrochemical reaction of a gold thin film.

As shown in FIG. 5, when an electrical signal is applied to the drug reservoir and the gold thin film is dissolved through an electrochemical reaction, it can be confirmed that a drug release channel opens.

[symbol]

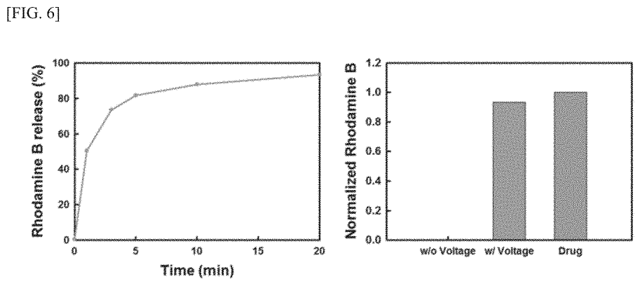

In addition, FIG. 6 shows a content of a model drug released by a drug delivery system.

As shown in FIG. 6, it can be conformed that, when no electrical signal was applied to a drug reservoir, the model drug is scarcely released and when an electrical signal is applied, most of the loaded model drug is released.

Thus, it can be confirmed that the release of a drug by the drug delivery system can be precisely controlled through an electrical signal.

Eventually, a drug for treating a dry eye syndrome is loaded into in the drug reservoir of the drug delivery system instead of the model drug, a change in current of a sensor by a dry eye syndrome biomarker is analyzed, and an electrical signal is transmitted, thereby releasing the drug of the drug delivery system.

### INDUSTRIAL APPLICABILITY

A contact lens for diagnosing and treating a dry eye syndrome according to the present invention can diagnose a dry eye syndrome through a change in current caused by an electric field effect when a dry eye syndrome biomarker is coupled to a probe. In addition, when the dry eye syndrome is diagnosed, an electrical signal can be generated, and a gold film blocking a drug reservoir can be dissolved by a current according to the electrical signal to adjust the release of a drug. Therefore, the contact lens according to the present invention can be effectively used for diagnosing and treating a dry eye syndrome.

1. A contact lens for diagnosing and treating a dry eye syndrome, comprising:
a dry eye syndrome biomarker detecting sensor; and
a drug reservoir,
wherein the dry eye syndrome biomarker detecting sensor and the drug reservoir are formed on a transparent substrate.

2. The contact lens of claim 1, wherein the contact lens is based on at least one selected from the group consisting of an elastomer such as a silicone elastomer, a silicone hydrogel, polydimethylsiloxane (PDMS), a polymer hydrogel of poly(2-hydroxyethyl methacrylate) (PHEMA), and poly(ethylene glycol) methacrylate (PEGMA).

3. The contact lens of claim 1, wherein the transparent substrate includes at least one selected from the group consisting of parylene C, polydimethylsiloxane (PDMS), a silicone elastomer, polyethylene terephthalate (PET), and polyimide (PI).

4. The contact lens of claim 1, wherein the dry eye syndrome biomarker includes at least one selected from the group consisting of a vascular endothelial growth factor (VEGF), lactoferrin, and lipocalin.

5. The contact lens of claim 1, wherein the dry eye syndrome biomarker detecting sensor is a field effect transistor biosensor.

6. The contact lens of claim 5, wherein the dry eye syndrome biomarker detecting sensor includes:
a source electrode and a drain electrode formed on the transparent substrate and spaced apart from each other by a certain distance;
a field effect transistor channel configured to connect the source electrode and the drain electrode; and
a probe attached onto the field effect transistor channel.

7. The contact lens of claim 6, wherein:
the source electrode and the drain electrode each independently include nanomaterials; and
the nanomaterials include at least one selected from the group consisting of zero-dimensional materials that are nanoparticles, one-dimensional nanomaterials that are nanowires, nanofibers, or nanotubes, and two-dimensional nanomaterials that are graphene, MoS₂, or nanoflakes.

8. The contact lens of claim 6, wherein the field effect transistor channel includes at least one selected from the group consisting of one-dimensional materials including silicon nanowires and two-dimensional materials including graphene or MoS₂.

9. The contact lens of claim 1, wherein the drug reservoir includes:
an electrode pattern including gold and formed on a portion of a surface of the transparent substrate; and
a drug well layer formed on the electrode pattern and including one or more drug weds with a shape that is recessed to face outward,
wherein:
perforations are formed in the transparent substrate; and
the electrode pattern surrounds the perforations.

10. The contact lens of claim 9, wherein a drug included in the drug well is a drug for treating a dry eye syndrome or includes a drug carrier configured to allow the drug for treating the dry eye syndrome to be released, and a drug-release-controlling material.
